(19) United States
(12) Patent Application Publication
(10) Pub. No.: US 2006/0110441 A1
(43) Pub. Date: May 25, 2006

(54) Lyophilized liposome formulations and method

(76) Inventors: Harry Wong, Palo Alto, CA (US); Yuanpeng Zhang, Cupertino, CA (US); Anthony Hei-Leung Huang, Saratoga, CA (US)

Correspondence Address: PERKINS COIE LLP, P.O. BOX 2168, MENLO PARK, CA, 94026, US

(21) Appl. No.: 11/261,983

(22) Filed: Oct. 28, 2005

Related U.S. Application Data
(60) Provisional application No. 60/623,393, filed on Oct. 28, 2004.

(51) Int. Cl. A61K 9/127; A61K 38/13; A61K 31/7048; A61K 31/7012; A61K 31/573; A61K 31/7072; A61K 31/55; A61K 31/4706; A61K 31/337; A61K 31/4196

(52) U.S. Cl. 424/450; 514/11; 514/27; 514/53; 514/217; 514/171; 514/313; 514/383; 514/449

(57) ABSTRACT

Formulations and methods for preparing a lyophilized composition comprising liposomes comprised of an unsaturated lipid and a hydrophobic drug associated with the liposome, and a cryoprotectant in a solution at a selected concentration. The phase transition temperature of the lipid is greater than the freezing point of the solution at the selected concentration.

This application claims the benefit of U.S. Provisional Application No. 60/623,393 filed Oct. 28, 2004, which is incorporated by reference herewith in its entirety.

### BACKGROUND

Liposomes are closed lipid vesicles used for a variety of purposes, and in particular, for carrying therapeutic agents to a target region or cell by systemic administration of liposomes. Liposomes have proven particularly valuable to buffer drug toxicity and to alter pharmacokinetic parameters of therapeutic compounds. For example, doxorubicin, amphotericin B, and liposome products incorporating these compounds are commercially available.

The stability and effective storage of pharmaceutical liposome preparations are important aspects of liposome products. Namely, it is important that liposome preparations can be stored for extended periods of time under appropriate conditions without undue loss of the encapsulated agent or alteration in size of the liposomes or significant changes in other physical or chemical characteristics.

It is well known in the art that many liposome formulations, including those with phospholipids, cannot be stored for sufficiently long periods of time as aqueous suspensions because of hydrolysis of the lipids. Thus, long term storage of liposomes may require lyophilization of the liposome formulation. Lyophilization, also known as freeze drying, refers to the process whereby a substance is prepared in dry form by freezing and dehydration. Lipids composed of fatty acids containing one or more double bonds (e.g., dioleoyl phosphatidylcholine or egg phosphatidylcholine) are considered especially unstable as powders. These lipids are extremely hygroscopic as powders and will quickly absorb moisture and become gummy upon opening the container resulting in hydrolysis or oxidation of the material. Accordingly, these lipids are generally available dissolved in a suitable organic solvent, transferred to a glass container with a teflon closure, and stored at <−20° C. (www.avantilipids.com). Shelf life of phosphatidyicholines at −20° C. is about 3 months for polyene lipids, about 6 months for monoene lipids, and about 12 months for saturated lipids.

Important concerns for lyophilization of liposome formulations include damage to the liposomes during freezing and the subsequent stability of the liposomes. Liposomal stability during storage is generally the extent to which a given formulation retains its original structure, chemical composition, and size distribution (U.S. Pat. No. 5,817,334). Instability of the liposomes can occur, for example, when liposome size increases spontaneously upon standing as a result of fusion or aggregation of the liposomes. Therapeutic agents may leak from the liposomes during fusion. Further, the liposomes may fuse to large multilamellar lipid particles at room temperature. These large liposomes or aggregates may precipitate as sediment. Breakage of the liposomes during drying is also a common problem, especially when appropriate cryoprotectants are not used. Breakage of the liposome results in leakage or release of the encapsulated contents. Additionally, the process of fusion and aggregation of unilamellar vesicles may be accelerated when the liposomes are subjected to freeze-thawing or dehydration as evidenced by a study showing small unilamellar vesicles of egg phosphatidylcholine reverting to large multilamellar structures upon freezing and thawing (Strauss and Hauser, PNAS USA, 83:2422 (1986)).

A common method used to protect vesicle integrity during dehydration and freezing is to include a cryoprotectant, such as a sugar, in the liposome formulation (Harrigan, P. R. et al., Chemistry and Physics of Lipids, 52:139-149 (1990)). The cryoprotectant preserves the integrity of the liposomes and prevents vesicle fusion and loss of vesicle contents. U.S. Pat. No. 4,927,571 describes a liposome formulation containing doxorubicin which is reconstituted from a lyophilized form that includes between 1-10% of a cryoprotectant, such as trehalose or lactose.

In U.S. Pat. No. 4,880,635, a dehydrated liposome formulation is prepared by drying the liposomes in the presence of a sugar, where the sugar is present both on the inside and outside of the liposome bilayer membrane. Similarly, U.S. Pat. No. 5,077,056 describes a dehydrated liposome formulation which includes a protective sugar, preferably on both the internal and external liposome surfaces.

Other liposome formulations, such as DOXIL®, a liposomal formulation containing doxorubicin, are suspensions where the liposomes are not dehydrated for later reconstitution, but remain in suspension during storage. The suspension medium may include a sugar for protection from freezing damage.

However, efficient and stable loading of hydrophobic drugs into liposomes at high concentrations is a challenge. Maintaining the product characteristics of the pre-lyophilized liposomal formulation after lyophilization has been a difficult or impossible problem for most liposomal formulations. This invention identifies lipids and lyophilization conditions that can provide efficient and stable loading of hydrophobic drugs into liposomes that can be successfully lyophilized.

### SUMMARY

In one aspect the invention includes a lyophilized composition comprising liposomes comprised of an unsaturated lipid, a hydrophobic drug associated with the liposome, and a cryoprotectant in a solution at a selected concentration. The phase transition temperature of the lipid is greater than the freezing point of the solution at the selected concentration. In one embodiment, the phase transition temperature of the lipid is at least 1° C. greater than the freezing point of the cryoprotectant in the solution. In one embodiment, the liposome composition may be comprised of a lipid mixture that contains at least 10 mol % of at least one unsaturated lipid.

In one embodiment, the lipid is an unsaturated lipid. In a preferred embodiment, the lipid is selected from palmitoyl-oleoylphosphatidylcholine, oleoyl-palmitoylphosphatidylcholine, stearoyl-oleoylphosphatidylchonline, oleoyl-stearoylphosphocholine, and egg phosphatidylcholine.

In one embodiment, the cryoprotectant is a disaccharide selected from the group consisting of sucrose, maltose, trehalose, and lactose. In another embodiment, the cryoprotectant is a disaccharide having a concentration selected from 5%, 10%, 12%, 15%, 20%, and 25%.

In a specific embodiment, the lipid is palmitoyl-oleoylphosphatidylcholine and the cryoprotectant is sucrose.

In a further embodiment, the hydrophobic drug is selected from paclitaxel, etoposide, cyclosporin A, docetaxel, cephalomannine, camptothecin, bryostatin-1, plicamycin, fluorouracil, chlorambucil, acetaminophen, antipyrine, betamethasone, carbamazepine, chloroquine, chlorprothixene, corticosterone, and 1(2′,6′-difluorobenzoyl)-5-amino-3-(4′-aminosulfonylanilino)-1,2,4-triazole. In yet another embodiment, the hydrophobic drug is a lipophilic compound having a water solubility of ≦100 μg/mL.

In a second aspect, the invention comprises a method of preparing a lyophilized liposome composition comprising preparing a liposome composition comprised of an unsaturated lipid, a hydrophobic drug associated with the liposome, and a cryoprotectant at a selected concentration. In this aspect, the phase transition temperature of the lipid is greater than the freezing point of the cryoprotectant in solution at the selected concentration. The liposome composition is then lyophilized. In one embodiment, the liposome composition may be comprised of a lipid mixture that contains at least 10 mol % of at least one unsaturated lipid.

In another embodiment, the preparing step further includes selecting a lipid and selecting a concentration of cryoprotectant in the solution. The selecting steps achieve a phase transition temperature of the lipid that is at least 1° C. greater than the freezing point of the cryoprotectant in the solution.

In one embodiment, the lipid is selected from palmitoyl-oleoylphosphatidylcholine, oleoyl-palmitoylphosphatidylcholine, stearoyl-oleoylphosphatidylchonline, oleoyl-stearoylphosphocholine, and egg phosphatidylcholine.

In another embodiment, the cryoprotectant is a disaccharide selected from the group consisting of sucrose, maltose, trehalose, and lactose. In specific embodiments, the cryoprotectant is a disaccharide with a concentration selected from 5%, 10%, 12%, 15%, 20%, and 25%

In a specific embodiment, the lipid is palmitoyloleoylphosphatidylcholine and the cryoprotectant is sucrose.

In one embodiment, the hydrophobic drug is selected from paclitaxel, etoposide, cyclosporin A, docetaxel, cephalomannine, camptothecin, bryostatin-1, plicamycin, fluorouracil, chlorambucil, acetaminophen, antipyrine, betamethasone, carbamazepine, chloroquine, chlorprothixene, corticosterone, and 1 (2′,6′-difluorobenzoyl)-5-amino-3-(4′-aminosulfonylanilino)-1,2,4-triazole. In another embodiment, the hydrophobic drug is a lipophilic compound having a water solubility of ≦100 μg/mL.

### DETAILED DESCRIPTION

### I. DEFINITIONS

The terms below have the following meanings unless indicated otherwise.

“Cryoprotectant” refers to a compound suitable to protect against freezing damage. Preferred cryoprotectants include sugars (disaccharides and monosaccharides), glycerol and polyethylene glycol.

“Liposomes” are vesicles composed of one or more concentric lipid bilayers which contain an entrapped aqueous volume. The bilayers are composed of two lipid monolayers having a hydrophobic “tail” region and a hydrophilic “head” region, where the hydrophobic regions orient toward the center of the bilayer and the hydrophilic regions orient toward the inner or outer aqueous phase.

“Vesicle-forming lipids” refers to amphipathic lipids which have hydrophobic and polar head group moieties, and which can form spontaneously into bilayer vesicles in water, as exemplified by phospholipids, or are stably incorporated into lipid bilayers, with the hydrophobic moiety in contact with the interior, hydrophobic region of the bilayer membrane, and the polar head group moiety oriented toward the exterior, polar surface of the membrane. The vesicle-forming lipids of this type typically include one or two hydrophobic acyl hydrocarbon chains or a steroid group, and may contain a chemically reactive group, such as an amine, acid, ester, aldehyde or alcohol, at the polar head group. Included in this class are the phospholipids, such as phosphatidyl choline (PC), phosphatidyl ethanolamine (PE), phosphatidic acid (PA), phosphatidyl inositol (PI), and sphingomyelin (SM), where the two hydrocarbon chains are typically between about 14-22 carbon atoms in length, and have varying degrees of unsaturation. Also included within the scope of the term “vesicle-forming lipids” are glycolipids, such as cerebrosides and gangliosides. “Vesicle-forming lipids,” as used herein, specifically excludes sterols, such as cholesterol.

“Unsaturated lipid” refers to a vesicle forming lipid having at least one degree of unsaturation. Unsaturation refers to a carbon atom in the fatty acid chain bound to less than the maximum possible number of hydrogen atoms. In this instance, adjacent carbon atoms share a double, rather than single, bond. Exemplary unsaturated lipids include egg phosphatidylcholine, asymmetric lipids such as palmitoleoyl phosphatidylcholine, stearyoyl-oleoyl phosphatidylcholine, oleolyl-palmitoyl phosphatidylcholine, and oleoyl-stearoyl phosphatidylcholine, and symmetric lipids such as dipalmitoeoyl phosphatidylcholine, and dioleoyl phosphatidylcholine.

The terms “hydrophobic”, “lipophilic”, and “non-polar” are used interchangeably to describe molecules that are not appreciably soluble in water or other polar solvents.

“Hydrophilic polymer” as used herein refers to a polymer having moieties soluble in water, which lend to the polymer some degree of water solubility at room temperature. Exemplary hydrophilic polymers include polyvinylpyrrolidone, polyvinylmethylether, polymethyloxazoline, polyethyloxazoline, polyhydroxypropyloxazoline, polyhydroxypropyl-methacrylamide, polymethacrylamide, polydimethyl-acrylamide, polyhydroxypropylmethacrylate, polyhydroxyethylacrylate, hydroxymethylcellulose, hydroxyethylcellulose, polyethyleneglycol, polyaspartamide, copolymers of the above-recited polymers, and polyethyleneoxide-polypropylene oxide copolymers. Properties and reactions with many of these polymers are described in U.S. Pat. Nos. 5,395,619 and 5,631,018.

“Freezing damage” refers to any one of a number of undesirable effects upon exposure of a liposome formulation to a temperature sufficient to cause freezing with one or more of the undesirable effects. Such effects include an increase in particle size due to aggregation and/or fusion of vesicles, and loss of encapsulated agent. The actual temperature which can cause onset of such an effect will vary according to the liposome formulation, e.g., the cryoprotectant, the type of lipids and other bilayer components, as well as the entrapped medium and therapeutic agent. Sometimes freezing damage is less at very cold freezing temperatures. This damage may be even less if the rate of freezing and thawing is fast. A temperature which results in freezing damage is typically a temperature lower than 0° C., more typically a temperature lower than −5° C., even more typically lower than −10° C. It will be appreciated that the freezing damage may decrease at the lower temperatures.

“Stability” as referring to lyophilized liposomes includes retention of the liposome structure, chemical composition, and/or size distribution.

Abbreviations: PC: phosphatidylcholine; PG: phosphatidylglycerol; PS: phosphatidylserine; PA: phosphatidic acid; POPC: palmitoyloleoyl phosphatidylcholine; EPC: egg phosphatidylcholine; DOPC: dioleoyl phosphatidylcholine; SOPC: stearyoyl oleoyl phosphatidylcholine; OPPC: oleolyl palmitoyl phosphatidylcholine; OSPC: oleoyl stearoyl phosphatidylcholine; DOPG: dioleoyl phosphatidylglycerol; DSPC: distearoyl phosphatidylcholine; PEG: polyethylene glycol.

### II. LIPOSOME FORMULATIONS

The present invention is directed to a liposome formulation having enhanced cryprotection properties for lyophilization. Preferably, the liposome formulation has increased protection from damage as a result of freezing. The liposomes in the formulation are primarily comprised of vesicle-forming lipids having at least one degree of unsaturation and include an associated therapeutic agent that is at least partially hydrophobic. It should be noted that lipid mixtures comprising at least one type of unsaturated lipid are suitable for the liposome formulations. Preferably, the lipid mixture contains at least 10 mol % of at least one unsaturated lipid. The liposome formulation may further comprise a cryoprotectant. These components will now be described.

A. Lipid

The lipids included in the bilayer of the present invention are generally vesicle-forming lipids having at least one degree of unsaturation. In exemplary embodiments, the vesicle-forming lipid has at least 1, 2, 3, 4, 5, or 6 degrees of unsaturation. It will be appreciated for lipids with asymmetric fatty acids, only one chain need be unsaturated, however, both chains may be unsaturated. It will be appreciated that lipid mixtures including at least one type of vesicle-forming lipid having at least one degree of unsaturation are contemplated for use. In some embodiments, the lipid mixture may include one or more unsaturated lipids and one or more saturated lipids. Preferably, the lipid mixture contains at least 10 mol % of at least one unsaturated lipid.

As seen in Table 1, lipids having at least one degree of unsaturation generally have a lower fluid/gel phase transition temperature than saturated lipids. The phase transition temperature (Tₘ) is the temperature required to induce a change in the physical state of the lipid from the generally ordered gel phase, where the hydrocarbon chains are fully extended and closely packed, to the disordered liquid crystalline phase, also called the fluid phase, where the hydrocarbon chains are randomly oriented and fluid. Processes for measuring the phase transition temperature of lipids are known in the art and include differential scanning calorimetry, nuclear magnetic resonance, x-ray diffraction, Fourier-transform infra-red spectroscopy, and fluorescence spectroscopy (Toombes et al.). Additionally, phase transition temperatures of many lipids are tabulated in a variety of sources, such as the Avanti Polar Lipids catalogue and Lipid Thermotropic Phase Transition Database (LIPIDAT, NIST Standard Reference Database 34). It will be appreciated that the exact Tₘ measured for a lipid will depend on the method of measurement.

Several factors are known to directly affect the phase transition temperature including hydrocarbon length, unsaturation, charge, and the headgroup species. Without being limited to the theory, as described below, introducing a double bond into the acyl group is thought to put a “kink” in the chain which requires much lower temperatures to induce an ordered packing arrangement (ntri.tamuk.edu/cell/lipid.html)

The carbon chain of a lipid comprising saturated fatty acids is more or less straight, without major bends. In contrast, an unsaturated fatty acid may take one of two forms at the double bond. In the cis form, the chain bends at an angle of about 30°, producing a “kink”. In the trans form, the chain is doubly bent so that the chain continues in the same direction without a pronounced kink, after the double bond. The kink of the cis form affects the packing of unsaturated fatty acid chains, resulting in more disordered, and consequently more fluid, bilayers (ntri.tamuk.edu/cell/lipid.html).

In one embodiment, the vesicle-forming lipids are selected to achieve a specified degree of fluidity to control the stability of the liposome in serum and to control the rate of release of the entrapped agent in the liposome. Lipid fluidity is achieved by incorporation of a relatively fluid lipid, typically one having a lipid phase with a relatively low gel-to-liquid-crystalline phase transition temperature, e.g., at or below body temperature, more preferably, at or below room temperature. Preferably, the unsaturated lipids of the present invention are in the fluid phase at room temperature (preferably about 15° C. to about 32° C., more preferably about 18° C. to about 26° C., typically about 22° C.). It swill be appreciated that the lipid phase transition temperature may be changed or manipulated to some degree by varying the conditions, such as pH, the buffering reagent, the ionic strength, the presence and amount of the therapeutic agent, and the presence of varying amount of miscible lipids having different phase transition temperatures. A comprehensive database LIPIDAT (www.lipidat.chemistry.ohio-state.edu) is available for information on lipid thermodynamics for most lipids.

TABLE 1
Phase Transition Temperatures for Unsaturated and Saturated Lipids

| Lipid (hydrated) | Chains/Backbone | Tₘ (° C.)¹ |
|---|---|---|
| EPC | — | −10 |
| | | (Crowe et al.) |
| POPC | 16:0-18:1 | −2 |
| DOPC | 18:1c9 | −20 |
| DOPG | 18:1 | −18 |
| DOPE | 18:1 | −16 |
| palmitoyl docosahexaenoyl PC | 16:0-22:6 | −27 |
| oleoyl palmitoyl PC | 18:1c9-16:0 | −9 |
| palmitoyl oleoyl PC | 16:0-18:1c9 | −5 to 3 |
| stearyol oleoyl PC | 18:1c9-18:0 | 8 to 13 |
| oleoyl stearyol PC | 16:0-18:1c9 | 5 to 13 |
| DOPG | 18:1 | −18 |
| dioleoyl PA | 18:1 | −8 |
| dioleoyl PS | 18:1 | −11 |
| dioleoyl phosphoethanolamine | 18:1c9 | −16 |
| dilinoleoyl phosphoethanolamine | 18:2 | −40 |
| DSPC | 18:0 | 55 |
| distereoyl PS | 18:0 | 68 |
| distereoyl PG | 18:0 | 55 |
| distereoyl PA | 18:0 | 75 |
| disteroyl phosphoethanolamine | 18:0 | 74 |

¹Avanti Polar Lipids (www.avantilipids.com) or LIPIDAT database except where noted

As further discussed further below, the therapeutic agent associated or entrapped within the liposome is a hydrophobic agent. Hydrophobic agents or drugs entrapped in a liposome are generally localized in the bilayer. Thus, the rigidity or fluidity of the lipid and the liposome influences the amount drug able to be entrapped in the bilayer as the lipids must be fluid enough to allow room for the drug. It will be appreciated that the degree of hydrophobicity and the size of the agent will affect the degree of fluidity that is required for localization in the bilayer. Generally, lipids that are more fluid are preferable for entrapping hydrophobic therapeutic agents as the fluidity of the lipids allow the drug to localize in the bilayer.

As noted above, unsaturated lipids for use in the present invention are preferably in the fluid phase at room temperature. Preferably, the unsaturated lipids have a phase transition temperature Tₘ for the hydrated lipid greater than about 0° C. to about −20° C. This range relates to the observed range for water freezing and crystallizing. It will be appreciated that where the cryopreservative and/or liposome suspension has a lower or higher freezing point, the preferred range will shift higher or lower accordingly. In this embodiment, the phase transition temperature of the lipid is preferably higher than the freezing point of the suspension. It will further be appreciated that lipids with a lower Tₘ could become useful as carriers of hydrophobic drugs for lyophilization when combined with a cryopreservative that lowers the freezing point of the suspension below the Tₘ of the lipid. DOPC, for instance, has a Tₘ of about −20° C.; however, DOPC is suitable in the present invention when used with a cryopreservative that lowers the freezing point of the liposome suspension below about −20° C.

The vesicle-forming lipids are preferably those having two hydrocarbon chains, typically acyl chains, and a polar head group. Included in this class are the phospholipids, such as phosphatidylcholine (PC), phosphatidylethanolamine (PE), phosphatidic acid (PA), phosphatidylinositol (PI), and sphingomyelin (SM), where the two hydrocarbon chains are typically between about 14-22 carbon atoms in length, and have varying degrees of unsaturation. Also included in this class are the glycolipids, such as cerebrosides and gangliosides. A preferred vesicle-forming lipid is a phospholipid. It is noted that lipids such as cholesterol, cholesterol derivatives, such as cholesterol sulfate and cholesterol hemisuccinate, and related sterols are generally considered unsuitable for use with the liposomes of the present invention as they lend rigidity to the bilayer and decrease the loading of hydrophobic therapeutic agent into the liposome. It will be appreciated that small amounts of sterols may be included where the rigidity of the liposome does not decrease loading of the therapeutic agent beyond acceptable limits, i.e. below a therapeutic dose.

More generally, “vesicle-forming lipid” is intended to include any amphipathic lipid having hydrophobic and polar head group moieties, and which (a) by itself can form spontaneously into bilayer vesicles in an aqueous medium, as exemplified by phospholipids, or (b) is stably incorporated into lipid bilayers in combination with phospholipids, with its hydrophobic moiety in contact with the interior, hydrophobic region of the bilayer membrane, and its polar head group moiety oriented toward the exterior, polar surface of the membrane. In a preferred embodiment, the liposome comprises at least between about 20-100 mole percent vesicle-forming lipids. The lipids of the invention may be prepared using standard synthetic methods. The lipids of the invention are further commercially available (Avanti Polar Lipids, Inc., Birmingham, Ala.).

The liposome can optionally include at least one vesicle-forming lipid derivatized with a hydrophilic polymer, as has been described, for example in U.S. Pat. No. 5,013,556, incorporated herein by reference. Including such a derivatized lipid in the liposome formulation may form a surface coating of hydrophilic polymer chains around the liposome. The hydrophilic polymer chains are effective to increase the in vivo blood circulation lifetime of the liposomes when compared to liposomes lacking such hydrophilic polymers.

Preparation of vesicle-forming lipids derivatized with hydrophilic polymers has been described, for example in U.S. Pat. No. 5,395,619, incorporated herein by reference. Preparations of liposomes including such derivatized lipids typically include between 1-20 mole percent of such a derivatized lipid included in the liposome formulation. A preferred hydrophilic polymer chain is polyethyleneglycol (PEG), preferably as a PEG chain having a molecular weight between 500-10,000 daltons, more preferably between 1,000-5,000 daltons. Vesicle-forming lipids suitable for derivatization with a hydrophilic polymer include any of those lipids listed above, and, in particular phospholipids. The hydrophilic polymer may further be attached to the lipid a releasable or cleavable linkage i.e. by a dithiobenzyl linkage as described in U.S. Pat. No. 6,342,244, incorporated herein by reference.

The vesicle-forming lipids of the bilayer may optionally include a targeting ligand surface group. “Targeting ligand” refers to a material or substance which promotes targeting to tissues, receptors and/or intracellular bodies. The targeting ligand may further be a ligand capable of being internalized by a cell. These targeting ligands optimize internalization of a therapeutic agent into the cytoplasm of a cell by specifically binding to the cell. The targeting ligand may be synthetic, semi-synthetic, or naturally-occurring. Such ligands are known in the art and described in U.S. Pat. No. 6,586,002 and co-owned U.S. Application No. 2003/0198665, both of which are incorporated herein by reference. Methods of attaching the ligand directly to the polar head group of the lipid are known in the art and described in U.S. Pat. Nos. 5,059,421, and 5,399,331. Where the liposome includes lipids derivatized to include a hydrophilic polymer, the ligands can be attached to the distal end of the hydrophilic polymer. Methods of covalently attaching the ligand to the free distal end of a hydrophilic polymer chain includes activating the free, unattached end of the polymer for reaction with a selected ligand, and in particular, the hydrophilic polymer polyethyleneglycol (PEG) and are widely known (Allen, T. M., et al., Biochemicia et Biophysica Acta 1237:99-108 (1995); Zalipsky, S., Bioconjugate Chem., 4(4):296-299 (1993)). It will be appreciated that the liposome may contain ligands attached to the distal end of the hydrophilic polymer and/or the polar head group of the lipid.

B. Therapeutic Agent

In one aspect of the invention, the bilayer formed of the lipids described above includes an entrapped therapeutic agent. By “entrapped” it is meant that a therapeutic agent is entrapped in the liposome lipid bilayer spaces and/or central compartment, is associated with the external liposome surface, or is both entrapped internally and externally associated with the liposomes.

In a preferred embodiment, the therapeutic agent is a hydrophobic agent, that is, an agent that is poorly or not soluble in an aqueous solution. Hydrophobic compounds are typically localized in the bilayer core or at the membrane interface.

The aqueous solubility of a compound can generally be determined by LogP measurements. These measurements show the degree to which the compound is partitioned between water and octanol (or other non-miscible solvent). Generally, a higher LogP number means that a compound is less soluble in water. The LogP of neutral immiscible liquids run parallel with their solubilities in water; however for solids, solubility also depends on the energy required to break the crystal lattice. The following equation has been suggested to relate solubility, melting point and LogP:
LogP=6.5−0.89(logS)−0.15 mpt

where S is the solubility in water in micromoles per liter (Bannerjee et al., Envir. Sci. Tech, 14:1227 (1980). Typically, a higher LogP number indicates the compound is poorly or not appreciably soluble in an aqueous solution. For example, paclitaxel is poorly water soluble at about 1 μM/L or 0.8 μg/mL and has a LogP of 7.4. LogP values for some exemplary hydrophobic agents are listed in Table 2. However, it will be appreciated that it is possible to have compounds with high LogP values that are still soluble on account of their low melting point. Similarly it is possible to have a compound having a high melting point with a low LogP where the compound is very insoluble. Some compounds having a LogP around zero may still have a very low water solubility, such as 1-(2′,6′-difluorobenzoyl)-5-amino-3-(4′-aminosulfonylanilino)-1,2,4-triazole (www.raell.demon.co.uk/chem/logp).

TABLE 2
LogP Values

| Compound | LogP |
|---|---|
| paclitaxel | 7.4 |
| etopside | 0.3 |
| cyclosporin A | 3.4 |
| docetaxel | 6.6 |
| cephalomannine | 6.0 |
| camptothecin | 1.9 |
| bryostatin-1 | 5.4 |
| plicamycin | 1.3 |
| fluorouracil | −0.8 |
| chlorambucil | 3.1 |
| acetaminophen | 0.34 |
| antipyrine | 2.5 |
| betamethasone | 1.9 |
| carbamazepine | 2.7 |
| chloroquine | 4.7 |
| chlorprothixene | 6.1 |
| corticosterone | 1.8 |

By way of comparison, paclitaxel has an aqueous solubility of 1 μM/L or 0.8 μg/mL, etopside has an aqueous solubility of 0.03 mg/mL, and cyclosporin A is 0.04 mg/ML soluble at 25° C. In a preferred embodiment, the therapeutic agent has a water solubility of ≦100 μg/mL

Agents contemplated for use in the formulations of the invention are widely varied, and include both therapeutic applications and those for use in diagnostic applications.

Therapeutic agents include natural and synthetic compounds having the following therapeutic activities: anti-arthritic, anti-arrhythmic, anti-bacterial, anticholinergic, anticoagulant, antidiuretic, antidote, antiepileptic, antifungal, anti-inflammatory, antimetabolic, antimigraine, antineoplastic, antiparasitic, antipyretic, antiseizure, antisera, antispasmodic, analgesic, anesthetic, beta-blocking, biological response modifying, bone metabolism regulating, cardiovascular, diuretic, enzymatic, fertility enhancing, growth-promoting, hemostatic, hormonal, hormonal suppressing, hypercalcemic alleviating, hypocalcemic alleviating, hypoglycemic alleviating, hyperglycemic alleviating, immunosuppressive, immunoenhancing, muscle relaxing, neurotransmitting, parasympathomimetic, sympathominetric plasma extending, plasma expanding, psychotropic, thrombolytic and vasodilating. Exemplary hydrophobic therapeutic agents include 1,2,4-triazole-3,5-diamine derivatives such as (1-(2′,6′-difluorobenzoyl)-5-amino-3-(4′-aminosulfonylanilino)-1,2,4-triazole), paclitaxel, doxorubicin, etopside, cyclosporin A, docetaxel, cephalomannine, camptothecin, bryostatin-1, plicamycin, fluorouracil, chlorambucil, acetaminophen, antipyrine, betamethasone, carbamazepine, chloroquine, chlorprothixene, corticosterone, zosuquidar, diltiazem, fluocortolone, griseofulvin, hydrocortisone, and lorazepam.

The therapeutic agent may further be an amphiphilic compound, which is a molecule that possesses both a hydrophilic and a hydrophobic part; and where at least a part of the compound is localized in the liposome bilayer.

The therapeutic agent may be incorporated in the liposome by any suitable method, including, but not limited to, (i) passive entrapment of a lipophilic compound by hydrating a lipid film containing the agent, (ii) loading an ionizable drug against an inside/outside liposome ion gradient, and (iii) loading against an inside/outside pH gradient. Other methods, such as reversed phase evaporation liposome preparation, are also suitable. Preferably, the liposomes are loaded by active drug loading methods including using an ion gradient such as an ammonium ion gradient as described in U.S. Pat. No. 5,192,549, incorporated herein by reference. It will be appreciated that hydrophobic drugs are typically loaded by passive entrapment.

It will be appreciated that the amount or concentration of hydrophobic drug that can be accommodated in the liposomes depends on drug/lipid interactions in the bilayer membrane.

It will further be appreciated that one or more therapeutic agents may be associated with the liposome. Contemplated embodiments include (i) two or more hydrophobic therapeutic agents localized in the bilayer and (ii) at least one hydrophobic agent localized in the bilayer and one or more hydrophilic agents entrapped within the aqueous inner space of the liposome.

C. Cryoprotectant

In one embodiment, the liposome formulation additionally includes at least one cryoprotectant. The cryoprotectant may serve to lower the freezing point of the formulation such that the Tₘ of the lipids of the liposome is reached (in the gel phase) before the freezing point of the formulation is reached. It will be appreciated that any dissolved substance added to the water will cause a freezing point drop. For every mole of nonelectrolytes dissolved in a kilogram of water in a dilute solution, the freezing point is reduced by approximately 1.86° C. The change in freezing point caused by the presence of a solute dissolved in an aqueous solution can be calculated from the equation:
T=(Kf)(m)(i)
where Kf is the molal freezing point depression constant (1.86° C./m for water), m is the molality of the solution, and i is the number of particles produced per formula unit.

The cryoprotectant serves to depress the freezing point of the formulation sufficiently to allow the lipids to reach the gel phase before the solution freezes or before significant ice crystals are formed during the freezing. It will be appreciated that the selected cryoprotectant should not have an eutectic or collapse temperature so low that the temperature during primary drying is lowered to cause the drying time to be overly extended. The cryoprotectant may further increase the Tₘ of the lipid to further separate the phase transition temperature from the formulation freezing temperature. It will be appreciated that the exact freezing point of the aqueous solution, with or without the cryoprotectant, will be dependent on the rate the solution is frozen.

In a preferred embodiment, the cryoprotectant is a monosaccharide or disaccharide sugar. In a more preferred embodiment, the cryoprotectant is a disaccharide. Suitable sugars include trehalose, maltose, sucrose, glucose, lactose, dextran, and aminoglycosides. It will be appreciated that the sugar may be used in various concentrations. Exemplary concentrations include, but are not limited to, 5%, 10%, 12%, 15%, 20%, and 25% inclusive. It will be appreciated that the concentration may be selected between 1% and 25%, or any concentration between these concentrations such as 3%. It will further be appreciated that more than one cryoprotectant may be used. In another embodiment, the cryoprotectant may be used in combination with other suitable protectants. An exemplary combination includes 3-4 K polyethylene glycol and 5% sucrose.

The cryoprotectant is included as part of the internal and/or external media of the liposomes. In a preferred embodiment, the cryoprotectant is included in both the internal and external media. In this embodiment, the cryoprotectant is available to interact with both the inside and outside surfaces of the liposomes membranes. Inclusion in the internal medium is accomplished by adding the cryoprotectant to the hydration solution for the liposomes. Inclusion of the cryoprotectant in the external medium is typically accomplished during one or more of the following operations: hydration, diafiltration, and/or dilution.

Any suitable concentration of cryoproteciant may be used in the present invention including about 5% to about 15% (w/v). A preferred cryoprotectant is 10% sucrose. It will be appreciated that the ratio of cryoprotectant to lipid may be more important than the concentration of the cryoprotectant. Preferably, the weight ratio of cryoprotectant to lipid is from about 0.5:1 at 200 mM lipid in 10% sucrose to about 100:1 at 1 mM lipid in 10% sucrose. Preferable ratios of lipid to cryoprotectant include 2:1 to 1:100. An exemplary embodiment includes about 175 mM lipid and 10% sucrose as cryoprotectant in a ratio of about 1.4:1.

### III. METHOD OF LYOPHILIZATION

As will be illustrated below, the liposomes of the present invention can be stably stored for relevant periods of time. Also, the liposome formulation of the present invention finds use especially for dehydration of the liposome formulation. In another embodiment, the liposome formulation finds use for lyophilization (freeze-drying) of the formulation. These dehydrated or lyophilized formulations are suitable for extended storage. The formulation is stably storable for at least about 1-24 months. In some embodiments the formulation is stably storable for about 3-12 months. In yet other embodiments, the formulation is stably storable for about 6-12 months.

As described above, the liposome formulation is formed by selecting an unsaturated lipid and a cryoprotectant such that the lipid has a fluid/gel phase transition temperature below room temperature, yet greater than the freezing point of the cryoprotectant solution. In one embodiment, the phase transition temperature of the selected lipid is higher than the freezing point of the formulation. In a preferred embodiment, the phase transition temperature of the selected lipid is higher than the freezing point of the formulation by at least 1° C. In other embodiments, the phase transition temperature of the selected lipid is higher than the freezing point of the formulation by at least 2, 3, 4, 5, 10 degrees Celsius, or more. In this manner, the lipid is in the fluid phase when in solution and provides sufficient fluidity for a hydrophobic drug to associate with and within the lipid bilayer. However, for lyophilization, the liposomes enter the gel phase before the formulation freezes, thus reducing or eliminating damage to the liposomes.

Liposomes of the present invention preferably find use in retaining a loaded hydrophobic drug during lyophilization and after storage. As described in Example 2, liposomes were prepared with unsaturated lipids, DOPC or POPC. DOPC has a Tₘ of about −20° C., which was similar to the freezing point of the aqueous medium (−20° C.). As noted above, POPC has a Tₘ of −2° C. Thus, for the liposomes prepared with the DOPC, the lipids are in the fluid phase during freezing of the formulation. In contrast, by selecting a lipid with a Tₘ above the freezing point of the formulation, POPC in this instance, the lipids are in the gel phase during freezing. After lyophilization and reconstitution, the % crystals in the aqueous medium was determined as shown in Table 3. The % crystals in the aqueous medium relates to the amount of drug leaked from the liposome, as the free drug is present in the aqueous medium as crystals or precipitate. Thus, a lower % crystal in the formulation after lyophilization relates to less leakage of the agent from the liposomes and a higher retention of the agent. As seen in Table 3, the DOPC liposome formulations showed a significant (about 25-45%) increase in MPD. After lyophilization and storage for one month at 40° C., the % crystals in the aqueous medium was further compared, as detailed in Example 3. Briefly, as seen in Table 4, the liposome formulations including DOPC had 6.88 to 7.87% crystal formation. In contrast, the liposome formulations including POPC had little or no crystals present in the aqueous medium. Thus, liposomes prepared according to the method of the invention were able to retain the loaded hydrophobic drug by a factor of at least 5 over the liposomes prepared with lipid having a lower Tₘ. Preferably, the liposome formulations of the present invention are able to retain the loaded hydrophobic drug by a factor at least 8, at least 10, or more over liposome formulations prepared with saturated lipids or with lipids having a Tₘ lower than the freezing point of the formulation. In a preferred embodiment, 70-100% of the drug is retained by the liposome formulations after lyophilization and storage for at least one month. In other embodiments, 80-100% or 90-100% of the drug is retained by the liposome formulations.

Liposomes of the present invention further find use in retaining their properties, especially mean particle diameter (MPD), after lyophilization. In experiments performed in support of the invention, liposomes prepared with POPC maintained a mean particle diameter (MPD) of about 100 nm (measured at 90°) after lyophylization and reconstitution as shown in Example 2. In contrast, the liposomes prepared with DOPC had a mean particle diameter of 500-1200 nm at 90° post reconstitution compared with about 100 nm before lyophylization (see Example 1). After lyophilization and reconstitution, the MPD of the liposomes prepared with a lipid having a lower Tₘ (i.e., DOPC) than the freezing point of the formulation increased 5 to 12 fold (500-1200% increase). The liposomes prepared with the unsaturated lipid selected according the present invention maintained a similar MPD before and after lyophilization.

A. Preparation of Liposomes

The liposomes may be prepared by a variety of techniques, such as those detailed in Szoka, F., Jr., et al., Ann. Rev. Biophys. Bioeng. 9:467 (1980), and specific examples of liposomes prepared in support of the present invention will be described below. Typically, the liposomes are multilamellar vesicles (MLVs), which can be formed by simple lipid-film hydration techniques. In this procedure, a mixture of liposome-forming lipids, including a vesicle-forming lipid derivatized with a hydrophilic polymer where desired, are dissolved in a suitable organic solvent which is evaporated in a vessel to form a dried thin film. The film is then covered by an aqueous medium to form MLVs, typically with sizes between about 0.1 to 10 microns. Exemplary methods of preparing derivatized lipids and of forming polymer-coated liposomes have been described in co-owned U.S. Pat. Nos. 5,013,556, 5,631,018 and 5,395,619, all of which are incorporated herein by reference. It will be appreciated that other types of liposomes may be useful in the present invention including SUVs and LUVs. The liposomes typically include about 5 mM to about 200 mM lipid concentration. In a preferred embodiment, the liposomes include about 175-200 mM, more preferably about 175 mM, of lipid. It will be appreciated that this range may vary depending on the amount of drug loaded, the size of the liposomes, and the medium used to prepare the liposomes.

As noted above, the therapeutic agent of choice can be incorporated into liposomes by standard methods, including (i) passive entrapment of a lipophilic compound by hydrating a lipid film containing the agent, (ii) loading an ionizable drug against an inside/outside liposome ion gradient, termed remote loading as described in U.S. Pat. Nos. 5,192,549 and 6,355, 268, both of which are incorporated herein by reference, and (iii) loading a drug against an inside/outside pH gradient. It will be appreciated that hydrophobic drugs are typically loaded by passive entrapment. If drug loading is not effective to substantially deplete the external medium of free drug, the liposome suspension may be treated, following drug loading, to remove non-encapsulated drug. Free drug can be removed, for example, by molecular sieve chromatography, diafiltration, dialysis, or centrifugation. In studies performed in support of the invention, a 1,2,4-triazole-3,5-diamine derivative (1 (2′,6′-difluorobenzoyl)-5-amino-3-(4′-aminosulfonylanilino)-1,2,4-triazole) that inhibits cyclin dependent kinase (CDK) activity was passively loaded to form liposomes comprised of POPC and DOPC as described in Example 1.

In one embodiment, the aqueous solution added to the dry film includes a cryoprotectant. In this manner, the cryoprotectant is present in the liposome internal aqueous space as well as in the aqueous medium. It will be appreciated that where it is desired for the cryoprotectant to be present only in the internal aqueous space of the liposomes, the external aqueous medium may be changed. It will further be appreciated that where it is desired that the cryoprotectant be present only in the external aqueous medium, the cryoprotectant may be added to the aqueous medium after hydration of the liposomes. It will be appreciated that the cryoprotectant may be added to achieve a desired molar ratio of cryoprotectant to lipid. In one embodiment, the cryoprotectant is present in a molar ratio of about 0-600 (based on 20% sucrose to 1 mM lipid) cryoprotectant to lipid.

After liposome formation, the vesicles may be sized to achieve a size distribution of liposomes within a selected range, according to known methods. The liposomes are preferably uniformly sized to a selected size range between 0.05 to 0.25 μm. MLVs or small unilamellar vesicles (SUVs), typically in the 0.04 to 0.08 μm range, can be prepared by sonication or homogenization of the liposomes. Homogeneously sized liposomes having sizes in a selected range can be produced, e.g., by extrusion through polycarbonate membranes or other defined pore size membranes having selected uniform pore sizes ranging from 0.07 to 0.5 microns, typically, 0.05, 0.07, 0.08, 0.1, 0.15, or 0.2 microns. The pore size of the membrane corresponds roughly to the largest size of liposomes produced by extrusion through that membrane, particularly where the preparation is extruded two or more times through the same membrane. The sizing is preferably carried out in the original lipid-hydrating buffer, so that the liposome interior spaces retain this medium throughout the initial liposome processing steps.

B. Lyophilization

Lyophilization includes freezing conditions that do not allow the water to freeze or the glass transition temperature of the formulation to be reached before the temperature drops below the phase transition temperature of the lipid.

Selecting a lipid with at least a single degree of unsaturation and with a phase transition temperature lower than room temperature and greater than the freezing point of the formulation as the major lipid in a liposomal formulation results in efficient and stable loading of hydrophobic drugs into liposomes that can be successfully lyophilized.

As described above, lyophilization usually refers to freezing the formulation followed by primary and, optionally, secondary drying. It will be appreciated that lyophilization, as used herein, may include only dehydration or only freezing of the formulation.

In the case of dehydration without prior freezing, if the liposomes being dehydrated have multiple lipid layers and, if the dehydration is carried out to an end point where there is sufficient water left in the preparation such that a substantial portion of the membranes retain their integrity upon rehydration, the use of the cryoprotectant may be omitted. In this embodiment, the preparation preferably contains at the end of the dehydration process at least about 2%, and most preferably between about 2% and about 5%, of the original water present in the preparation prior to dehydration.

The lyophilization of the formulation may be performed by any appropriate method. An exemplary method includes shelf-freezing in a freeze-dryer such as the Model 12K Supermodulyo available from Edwards High Vacuum (West Sussex, England). It will be appreciated that any available freeze-dryer finds use in the present invention. It will be appreciated that the rate of cooling will determine the apparent freezing point of the formulation. Suitable freezing rate include about 0.2-1° C./min. A preferred cooling rate is about 0.5° C./min. In another embodiment, the formulation is cooled from 0° C. to −40° C. or −50° C. in about 30 minutes.

After freezing, the formulation may be dried by suitable methods. In one embodiment, the formulation is dried in an available freeze dryer as noted above under a vacuum for an appropriate time. Exemplary conditions include primary drying the sample at about −35 to −50° C. for about 12-24 hours. Exemplary secondary drying conditions include drying at room temperature (about 25° C.) for about 5 to about 10 hours. It will be appreciated that other conditions and equipment are suitable for lyophilization.

It will be appreciated that drying methods other than lyophilization can be used in the invention, for example, spray, tray, and drum drying. The formulation may also be snap-frozen in an ethanol- or acetone-dry ice bath for at least 20 minutes, and lyophilized overnight at about −35 to about −50° C. under constant pressure overnight (Freezone 6, Labconco, Kansas City, Mo.).

The lyophilized “cake” may then be resuspended in an aqueous medium such as deionized water for use. Preferably, rehydration of the lyophilized formulation forms a suspension of liposomes which maintains the size distribution and morphology of the original liposomal suspension before freeze drying, and further maintains the drug to lipid ratio of the original liposomal suspension before freeze drying. In a preferred embodiment, about 50 to about 100% of the liposomes maintain the size distribution and/or drug to lipid ratio of the original formulation. More preferably, about 60, about 70, or about 80% of the liposomes maintain the size distribution and/or drug to lipid ratio of the original formulation.

### IV. EXAMPLES

The following examples illustrate but are in no way intended to limit the invention.

### Example 1

### Preparation of Liposomes

Liposomes comprised of POPC were loaded with 1(2′,6′-difluorobenzoyl)-5-amino-3-(4′-aminosulfonylanilino)-1,2,4-triazole by dissolving 1.03 grams of the drug with 37.9 grams lipid in 30 mL ethanol organic solvent by incubation with stirring at 50° C. for one hour until all of the drug and lipid were dissolved. In a separate container, 270 mL of hydration buffer (15 mM NaCl, 10 mM histidine, pH 6.1) was preheated to 50° C., followed by the addition of the lipid/ethanol solution in a fast and uniform rate. The lipid suspension was continuously agitated for one hour at about 50° C. The lipid suspension was then subjected to extrusion to produce LUVs by pushing through polycarbonate filters with step-down pore sizes (2 passes with 0.4 μm, 4 passes with 0.2 μm and 3 passes with 0.1 μm). The final liposome diameter was 101.6 nm and 106.3 nm, respectively, at 90° and 30° detector angles (Coulter N4MD submicron particles sizer). The ethanol was then removed by diafiltration by exchanging with 10 w/v % sucrose (8 volumes of 10 w/v % sucrose, 10 mM histidine, 15 mM NaCl, pH 6.0, A/G Technology Corporation diafiltration cartridge, MWCO 100k). At the end of diafiltration the formulation was concentrated, in order to maximize the drug concentration. With this method, about 3 to about 3.5 mg/mL of 1(2′,6′-difluorobenzoyl)-5-amino-3-(4′-aminosulfonylanilino)-1,2,4-triazole can be loaded into 175 to 200 mM liposomes.

### Example 2

### Lyophilization of DOPC and POPC Liposomes

Liposomes composed of DOPC or POPC were prepared as described in Example 1. The liposomes were then lyophilized under the following conditions.

| | |
|---|---|
| Shelf loading temperature | 0° C. |
| Product ramp time to freezing temperature | 5.5 hr |
| Shelf freezing temperature | −50° C. |
| Primary drying temperature | −5° C. |
| Primary drying pressure | 40 μ Hg |
| Primary drying time | 51 hr |
| Secondary drying temperature | 25° C. |
| Secondary drying pressure | 75 μ Hg |
| Secondary drying time | 67 hr |

The liposomes were subsequently reconstituted by replacing the water lost during lyophilization with water for injection to restore the original fill volume.

The MPD of the liposomes was measured as described in Example 1 after lyophilization and reconstitution. Further, the amount of crystals was measured in the external aqueous medium as a percentage of the amount of drug loaded in the liposomes. As the drug is hydrophobic, leakage of the drug from the liposome results in formation of a precipitate or crystals in the aqueous medium, which can be isolated by centrifugation of the samples and measured for the amount. The results of these studies are detailed in Table 3.

As seen in Table 3, the DOPC liposome formulations showed significant increase in MPD (in the range of 1000-2000 nm) when undiluted. When diluted 3×(2.5 mL fill volume), the MPD is significantly smaller (140-146 nm at 90° and 218-254 nm at 30°) than the undiluted, but still much larger than the MPD prior to lyophilization. In comparison, POPC liposomes showed little or no increase in MPD either diluted or undiluted when measured at both 30° and 90° at the two fill volumes (2.5 mL and 5 mL).

The diluted DOPC liposome formulations, however, showed significant drug loss from the liposomes probably as a result of drug crystal formation. As further seen in Table 3, about 22% of the drug loaded into the liposomes was lost upon reconstitution after lyophilization. With the POPC formulations, only about 1-4% of the drug loaded into the formulations was present in the medium after lyophilization and reconstitution.

TABLE 3
Mean Particle Diameter and % crystal formation in DOPC and POPC
liposome formulations at time zero

| | Fill | | Mean Particle | Mean Particle | |
|---|---|---|---|---|---|
| | volume | Dilution | Diameter at 90° | Diameter at 30° | % crystals |
| Lipid | (mL) | factor | (nm) | (nm) | in medium |
| DOPC | 2.5 | 3 x | 146 | 254 | 22.0 |
| DOPC | 2.5 | 3 x | 140 | 218 | 22.4 |
| DOPC | 5 | 1 x | 1751 | 1583 | |
| DOPC | 5 | 1 x | 1503 | 1677 | |
| POPC | 2.5 | 3 x | 108 | 143 | 3.82 |
| POPC | 5 | 1 x | 103 | 126 | 0.93 |

### Example 3

### Storage of Lyophilized DOPC and POPC Liposomes

Liposomes composed of DOPC or POPC were prepared as described in Example 1. The liposomes were then lyophilized under the following conditions:

| | |
|---|---|
| Shelf loading temperature | 0° C. |
| Product ramp time to freezing temperature | 4.9 hr |
| Shelf freezing temperature | −50° C. |
| Primary drying temperature | −25° C. |
| Primary drying pressure | 75 μ Hg |
| Primary drying time | 80.1 hr |
| Secondary drying temperature | 25° C. |
| Secondary drying pressure | 75 μ Hg |
| Secondary drying time | 25 hr |

The liposome suspensions were subsequently reconstituted by replacing the water lost during lyophilization with water for injection to restore the original fill volume.

The lyophilized liposome formulations were stored at 40° C. for one month. After one month, the formulation was rehydrated and the MPD and % crystals in the external medium, as a percentage of the amount of drug loaded in the liposomes, was determined as detailed in Table 4.

After storage, about 7-8% of the drug loaded into the liposomes was present in the external medium for the diluted DOPC liposome formulations. For the POPC formulations, none or very little of the drug that was loaded into the formulations leaked from the liposomes after lyophilization and reconstitution.

TABLE 4
Mean Particle Diameter at time zero and % crystal formation after one
month

| | | | Mean | | |
|---|---|---|---|---|---|
| | | | Particle | Mean Particle | % crystals in |
| | Fill | | Diameter | Diameter at 30° | medium |
| | volume | Dilution | at 90° (nm) | (nm) | t = 1 month |
| Lipid | (mL) | factor | t = 0 | t = 0 | at 40° C. |
| DOPC | 2.5 | 3 x | 120 | 170 | |
| DOPC | 5 | 3 x | 128 | 184 | |
| DOPC | 5 | 3 x | 128 | 185 | |
| DOPC | 5 | 3 x | | | 7.87 |
| DOPC | 5 | 3 x | | | 6.88 |
| DOPC | 10 | 3 x | 136 | 205 | |
| DOPC | 2.5 | 1 x | 1313 | 1090 | |
| DOPC | 5 | 1 x | 1523 | 1214 | |
| DOPC | 10 | 1 x | 1267 | 1031 | |
| POPC | 2.5 | 1 x | 96 | 123 | |
| POPC | 2.5 | 1 x | 97 | 120 | |
| POPC | 5 | 1 x | 96 | 119 | |
| POPC | 5 | 1 x | 97 | 115 | |
| POPC | 5 | 1 x | | | 0.00 |
| POPC | 5 | 1 x | | | 0.07 |
| POPC | 10 | 1 x | 95 | 117 | |
| POPC | 10 | 1 x | 96 | 110 | |

Although the invention has been described with respect to particular embodiments, it will be apparent to those skilled in the art that various changes and modifications can be made without departing from the invention.

It is claimed:

1. A lyophilized composition comprising:
liposomes comprised of an unsaturated lipid and a hydrophobic drug associated with the liposome; and
a cryoprotectant in a solution at a selected concentration;
wherein a phase transition temperature of the lipid is greater than a freezing point of the solution at the selected concentration.

2. The composition of claim 1, wherein the phase transition temperature of the lipid is at least 1° C. greater than the freezing point of the cryoprotectant in the solution.

3. The composition of claim 1, wherein the lipid is selected from the group consisting of palmitoyl-oleoylphosphatidylcholine, oleoyl-palmitoylphosphatidylcholine, stearoyl-oleoylphosphatidylcholine, oleoyl-stearoylphosphocholine, and egg phosphatidylcholine.

4. The composition of claim 1, wherein the cryoprotectant is a disaccharide selected from the group consisting of sucrose, maltose, trehalose, and lactose.

5. The composition of claim 1, wherein the lipid is palmitoyl-oleoylphosphatidylcholine and the cryoprotectant is sucrose.

6. The composition of claim 1, wherein the cryoprotectant is sucrose with the concentration selected from 5%, 10%, 12%, 15%, 20%, and 25%.

7. The composition of claim 1, wherein the hydrophobic drug is selected from paclitaxel, etoposide, cyclosporin A, docetaxel, cephalomannine, camptothecin, bryostatin-1, plicamycin, fluorouracil, chlorambucil, acetaminophen, antipyrine, betamethasone, carbamazepine, chloroquine, chlorprothixene, corticosterone, and 1(2′,6′-difluorobenzoyl)-5-amino-3-(4′-aminosulfonylanilino)-1,2,4-triazole.

8. The composition of claim 1, wherein the hydrophobic drug has a water solubility of <100 μg/mL.

9. The composition of claim 1, wherein said liposomes are comprised of a lipid mixture that contains at least 10 mol % of at least one unsaturated lipid.

10. A method of preparing a lyophilized liposome composition comprising:
preparing a liposome composition comprised of an unsaturated lipid, a hydrophobic drug associated with the liposome, and a cryoprotectant at a selected concentration, a phase transition temperature of the lipid being greater than a freezing point of the cryoprotectant at the selected concentration; and
lyophilizing the liposome composition.

11. The method of claim 10, wherein said preparing further includes:
selecting a lipid or a lipid mixture; and
selecting a concentration of the cryoprotectant in a solution;
whereby said selecting steps achieve a phase transition temperature of the lipid or the lipid mixture that is at least 1° C. greater than the freezing point of the cryoprotectant in the solution.

12. The method of claim 10, wherein said lipid is selected from the group consisting of palmitoyl-oleoylphosphatidylcholine, oleoyl-palmitoylphosphatidylcholine, stearoyl-oleoylphosphatidylcholine, oleoyl-stearoylphosphocholine, and egg phosphatidylcholine.

13. The method of claim 10, wherein the cryoprotectant is a disaccharide selected from the group consisting of sucrose, maltose, trehalose, and lactose.

14. The method of claim 10, wherein the lipid is palmitoyl-oleoylphosphatidylcholine and the cryoprotectant is sucrose.

15. The method of claim 10 wherein the cryoprotectant is sucrose with the concentration selected from 5%, 10%, 12%, 15%, 20%, and 25%.

16. The method of claim 10, wherein the hydrophobic drug is selected from paclitaxel, etoposide, cyclosporin A, docetaxel, cephalomannine, camptothecin, bryostatin-1, plicamycin, fluorouracil, chlorambucil, acetaminophen, antipyrine, betamethasone, carbamazepine, chloroquine, chlorprothixene, corticosterone, and 1(2′,6′-difluorobenzoyl)-5-amino-3-(4′-aminosulfonylanilino)-1,2,4-triazole.

17. The method of claim 10, wherein the hydrophobic drug has a water solubility of <100 μg/mL.

18. The method of claim 10, wherein said liposome composition is comprised of a lipid mixture that contains at least 10 mol % of at least one unsaturated lipid.
